(19) United States
(12) Patent Application Publication
(10) Pub. No.: US 2003/0138468 A1
(43) Pub. Date: Jul. 24, 2003

(54) Skin protective compositions

(76) Inventor: Armando R. Lopez, Mt. Kisco, NY (US)

Correspondence Address: Evelyn M. Sommer, 30th Floor, 825 Third Avenue, New York, NY, 10022, US

(21) Appl. No.: 10/051,994

(22) Filed: Jan. 22, 2002

(51) Int. Cl. A61K 7/00

(52) U.S. Cl. 424/401

(57) ABSTRACT

Skin protective compositions for topical use to prevent contact and passage to the skin of toxic chemicals and irritants found in the cosmetic industry, comprising a cosmetic vehicle or base and a surfactant for easy and ready removal from the skin with water. Methods of making and using such compositions are also disclosed.

The present invention relates to protective skin compositions, more particularly to protective skin compositions useful for forming a protective film on the skin after topical application, to provide an effective barrier function towards toxic and irritating materials for an extended period of time. The compositions provided in the form of creams, ointments, gels and films are readily washed off with water.

The present invention relates to skin protective compositions, in particular to topical compositions that can prevent the contact and access of toxic chemicals and irritants to the skin, for extended periods of time. These compositions will not interfere with the normal physiological functioning of the skin. These skin protectors or barrier compositions can be prepared as gels, creams, ointments and films. In addition to the protective barrier function of these compositions, skin enhancing and protective agents can be added, for instance, conditioners, cleansing agents can be included for delivery to the skin. Biologically active agents such as vitamins, antioxidants or herbal or plant products can also be included. These barrier compositions are readily removed with water. The barrier compositions can have their removability enhanced by the presence of surfactants and preferably a lathering surfactant.

### BACKGROUND OF THE INVENTION

The toxic and irritating chemicals of concern are those peculiar to the cosmetic industry and more specifically those used in the hair dressing industry and include agents used for changing the shape of hair; permanent waving, temporary waving and hair straightening. Also included are agents for changing hair color; bleaching, permanent hair coloring, semi hair coloring and tinting.

For permanent waving of hair (cold wave) dual solutions are required which are used separately and successively:

1. An alkaline thioglycolate solution (pH 8-10) waving fluid.

2. An acid peroxide solution (fixation and neutralizing fluid.) The hair is pretreated with an alkaline shampoo to make the hair more permeable to the waving fluid. The alkaline reaction swells the hair to almost 150% of its original size, which promotes the action of the thioglycolate. Even the strong bridges, S-S bonds, of the keratin filaments are broken. The thioglycolate solution is removed (blotting) and the second acid fluid is applied to the hair. The acid reaction of this solution stops the action of the residual thioglycolate and the peroxides restore the S-S bridges

Allergic reactions to thioglycollic acid, its salts and to other ingredients of these preparations are known to occur. In most cases the unwanted effects accompanying these procedures are of an irritant nature. The range of toxicities reported for the permanent wave composition of the type described include:

U.S. Manufactures file 1.7% 144/8399

U.S. Hospital file 5.2% 36/698

For straightening hair, three types of chemical relaxers are used; while they work quickly they are capable of causing skin irritation. The incidence of irritating skin reactions has been reported to be as high as 8%.

1. alkaline straighteners—These work the fastest, within 8 to 10 minutes, but they are also the most caustic. They are primarily used professionally although some alkali products, containing NaOH, are available for home use).

2. thioglycolate straighteners—These also are intended for use by professionals. They work in 10-15 minutes and must be neutralized with an oxidizer.

3. bisulfite straighteners—These are more effective than the thioglycolates and almost as effective as the alkaline straighteners. They are left on the hair for almost 15 minutes and must be neutralized. They are used professionally and also have the largest share of the home market.

In the actual hair straightening process a dual solution is used:

1. Straightening fluid which contains one of the following agents: Na(K)OH, sodium bisulfite or ammonium thioglycolate.

2. for NaOH a nonalkaline shampoo is sufficient For sodium bisulfite sodium bicarbonate is used; for ammonium thioglycolate hydrogen peroxide is used.

The incidence of hair straightener toxicity as reported in the U.S. Manufactures{s file is 3% (248/8399)

Hair Coloring—Hair color may be altered on a permanent, semi permanent or temporary basis.

1. Hair color restorers—are hair dressings which gradually (several days) darken gray hair to a brownish black color. The active ingredients, present in about a 1% concentration, are lead acetate and colloidal sulfur or sodium thiosulfate that form lead sulfide on the hair surface. Hair restorers contain lead and may contain silver in place of the lead. The lead or silver coat the hair and react with its protein to leave the permanent color of the particular metal used. These products are particularly popular with men. Lead and less often silver are known a known to represent toxic substances.

Tints are the most commonly used permanent dyes. They start out as colorless chemicals, usually combinations of phenol compounds and other agents that take on color when peroxide is added to the mix just before application to the hair. Tints are also known as oxidation dyes, because they must be oxidized by peroxide to couple the colorless intermediate chemicals. These two components are mixed just before use. Two separate and different reactions occur after the application of the mixture; the peroxide bleaches the hair pigment (melanin). The other occurring reaction is a complex one and results in the formation of the new desired color. This procedure takes about 15 minutes and when sufficient new color has formed the product is washed off.

The propensity for allergic reactions to these dyes, for example, toluene-2-5-diamine, 4-methoxy-phenylene diamine, exists and there is a potential for non-allergic skin reactions is possible. Permanent hair coloring preparations all carry warning instructions for the possibility and means for preventing such toxic reactions. A problem peculiar to these preparations is the staining of the skin that can occur while applying the reagents.

Bleaches—are essentially peroxide combined with a variety of stabilizers to prevent its decomposition and accelerators to improve the peroxide's efficiency. The oxidation of the hair with peroxide is performed as the first step to achieve the desired lighter tone. A second step follows by application of a toner. Allergic reactions may occur with bleaches as well as toners; additionally, the alkalinity of the bleaching solution may produce chemical skin burns. The incidence of side effects reported for hair colors:

U.S. Manufacturers file 14.1% 1187/8399

U.S. Hospital file 14.9% 104/698

Semi-permanent Hair Coloring—This method of hair coloring uses dyes that are shampooed into the hair and left in place for about a half hour to be oxidized slowly by the air. Most commonly non-oxidizing dyes are used that simply diffuse into the hair. They are called semi-permanent because while they penetrate the hair shaft, they wash out after 5 or 6 shampoos. They can change shaded within a narrow range of the individuals natural color. Like tints, semi-permanent dyes produce allergic reactions. One of the paradoxes of modern hair care is that the cosmetic procedures used damage the hair structure. The procedures and agent utilized for changing the shape and color of hair involve irritating and potentially dangerous substances. These substances are able biochemically to alter hair (chemically consisting mostly of keratin) by altering the molecular structure of the keratin. The same substance, keratin, is found as a major constituent of the upper layer of the skin. When its chemical identity is compromised by incidental cosmetic contamination during hair treatment a number of serious consequences may result; notably dermatitis which is defined as an inflammatory state of the skin when caused by external agents. The skin changes noted are redness of the skin (erythema) caused by engorgement and dilatation of the small blood vessels which is usually accompanied by swelling (edema), the result of fluid leaving the damaged blood vessels and accumulating in the tissue. If the swelling is severe, blisters form. Avoidance of the irritating material is not always practical so that protection becomes the best therapeutic approach, Hair preparations in general, including hair dyes, relaxants and waving preparations are responsible for a large number of allergic states. As for the types of ingredients that cause the allergic reactions, fragrances are responsible for the greatest number, preservatives are next followed by phenylenediamine found in hair dyes, and the hair waving ingredient glyceryl monothioglycolate can be mentioned In addition to the irritating and allergy inducing potential of these chemicals is the additional propensity for the dyes and to tints used, to stain the face, ears and necks of the individual whose hair color is being altered. Heretofore headbands made of woven or non woven materials have been used as have barrier materials such as petroleum jelly and the like.

Although there have been advances in the protection of skin from the harmful effects of cosmetics, the harmful effects of such hair cosmetics applications continues unabated; this becomes apparent in the warning labels on all hair coloring preparations as well as other hair cosmetics. Barrier creams, ointments and gels that protect the skin from chemicals, water soluble chemicals, as well chemicals soluble in oil represent the preferred preventative treatment. The need for a barrier composition that is readily applied, easily removed, without unacceptable odor or texture, and that is consumer and professionally acceptable, is readily apparent. Many of the commercially available topical barrier formulations or those prepared by cosmetologists in their own establishments consist of an oil based carrier such as; petrolatum or liquid petrolatum which are insoluble in water. Such compositions tend to be greasy, tacky, often difficult to remove and may permanently stain clothing,

Accordingly, it is an object of the present invention to provide compositions for topical application to the skin providing a barrier for toxic agents utilized in the hair dressing industry, which are readily removable with water.

It is another object of the invention to provide a barrier composition containing a surfactant. It is yet another object of the invention to provide a barrier composition containing a lathering surfactant. It is another object of the invention to provide such barrier compositions which additionally contain at least one agent for conditioning the skin. It is another object of the invention to provide such compositions comprising a surfactant and a conditioning component. It is another object of this invention to include in any of the above protective, barrier compositions one or several biologically active agent(s) to enhance the physiologic condition of the skin; such agents could include vitamins, proteins, complex carbohydrates (such as hyaluronic acid), antioxidants and plant products. This and other objects of the invention will become apparent in light of the following disclosure It has been surprisingly found in accordance with the present invention that compositions can be developed to provide effective barrier protection and preferably in addition, conditioning in a convenient, inexpensive, easily applied and as easily removed composition having the desirable properties of a cream, gel, ointment or film. The present invention provides the convenience of not needing to use both a barrier composition and separate cleansing or removal composition as well as to preferably provide for delivery to the skin, at the same time of a conditioning component.

### SUMMARY OF THE INVENTION

This invention provides skin protective or barrier compositions, in particular, topical compositions that can prevent the contact and passage to the skin, of toxic chemicals and irritants found in the cosmetic industry, and particularly peculiar to the hair dressing industry and which compositions can be readily removed with water. The skin protectives of this application, accomplish this by placing a passive protective barrier between the skin and the environment which prevent access of chemicals to the epidermis (upper-most layer of the skin). Their ready removal is achieved by incorporating into the formulation a surfactant. The compositions of the invention include surfactants which may be lathering surfactants. The surfactants are present in an amount of 0.5-25% by weight of the composition so that upon wetting with water, the compositions are easily and completely removed from the skin.: The compositions of this invention may also include skin conditioning agents that enhance the appearance, manageability and health of the skin to which the barrier cream is being applied. The topical protective compositions can contain additional pharmaceutically, physiologically active agents. They can be compounded as suspensions, emulsions, ointments, gels or films or mixtures of the above. The skin protective compositions are compounded of pharmacologically inert agents but can have introduced into the composition bioactive substances that physiologically enhance and protect the skin, for example, vitamins, antioxidants, plant products, and moisturizers. The compositions of the invention comprise either a conventional cream, gel, film lotion or the like used in the cosmetic or pharmaceutical industry for topical administration to the skin for serving as a barrier to toxic chemicals comprising in addition to the cream, gel, film or lotion formulation a surfactant, preferably a lathering surfactant and optionally a conditioning agent. The surfactant facilitates the removal of the cream, gel, or film; only water being required. The conditioner component is useful for providing a conditioning benefit to the skin during the use of a hair dye, hair relaxant, hair waving composition and the like. As many possibilities exist for the choice of the functional ingredients for the protective skin compositions, endless variations can be made. Generally these compositions include a water insoluble or only slightly water soluble lipid. For the lotions the water/lipid ratio determines the result. Ratios of 7 to 9 result in fluid products. Most oil in water creams have ratios of 1 to 2; the oily water in oil creams preferably have ratios of 0.5 to 1. The gels are the conventional two phase systems consisting of a solid and a liquid in a more solid form than a sol. A typical formula for a cream or lotion preparation would be compounded of: 20-90% water, 1-5% polyol (e.g. sorbitol), 10-80% lipid (e.g. stearic acid, cetearyl alcohol, squalene), 2-5% surfactant (e.g. polysorbate 40, TEAoleate), 0.5% special moisturizer (e.g. polyamino sugar condensate), 0.3% preservative (e.g. methyl or propyl paraben). A typical basic formula for a gel is as follows: 50-60% water, ca 5% polyol (e.g. sorbitol), ca 2% thickener (gelling agent) e.g. carbomer 914, 10-30% organic solvent (e.g. ethanol), 0.5% surfactant (e.g.TEAlauryl sulfate), 0.2% preservative (e.g.methyl or propyl paraben) A typical preparation for an aerosol foam would utilize 90% of the above compositions plus the addition of about 10% propellant (e.g. chlorofluorocarbon 12 and 14). Another typical formula for a barrier cream follows: About 65% water, ca 25% polyol (e.g. glycerol), ca 25% active barrier ingredient (e.g. silicones, cyclomethicone, stearic acid), ca 5% surfactant (e.g. sodium lauryl sulfate, cocamidipropylbetaine), and about 0.3% preservative (e.g. bromonitropropanediol).

Other compositions for use in the cosmetic industry as skin protectants include silicone gel compositions. Known silicone gel compositions include those comprised of silicone oil and wax, silicone oil and silica, and silicone oil and, polyoxyalkylene containing organopolysiloxanes. One such protective cream formulation contains 18-40% of an alkali metal fatty acid salt having 8 to 18 carbon atoms, 5-20% of a polyol effective as an emollient, 1.0-10.0% surfactant, 0.5-8% of an alkali metal silicate and the balance water. A specific example of such a composition is an oil in water emulsion base comprised of an alkali metal fatty acid such as sodium palmitate or sodium taurate, the latter provide water and chemical repellency. Alkali-metal salts of saturated or unsaturated fatty acids having from 8 to 18 carbon atoms are preferred, e.g. include sodium laurate (C10), sodium myristate (C12), sodium palmitate (C14), and sodium oleate (C16). An anionic surfactant, ammonium laureth sulfate is included in the composition. An alkali-metal silicate is added to the composition in an amount to reduce the tackiness of the fatty acid component and to enhance the effectiveness of the topical barrier. The amount of alkali-metal silicate added is about 0.5-3% The alkali-metal silicate may be sodium silicate, potassium silicate or may comprise a siloxane oligomer or polymer such as polydimethylsiloxane. A polyol may also be added to maintain the proper viscosity. Polyols such as glycerin also act as emollients. Preferred polyols include glycerin, sorbitol, and propylene glycol. The polyol is preferably used in an amount of 5-20% of the composition. The balance is water. The resulting product is a cream. The composition may be packaged as a gel by combining approximately 85% of the cream formulation and 15% of a thickening such as kaolin. The composition in spray form is prepared by incorporating into the cream formulation 15% propellant, (e.g. propane/butane mixture, propane/isobutane mixture, fluorocarbon propellants or just isobutane). The gel, spray and cream compositions dry quickly on the skin and may form a film. Of particular interest for use in this invention are barrier creams, ointments or gels. Suitable barrier creams for use in this invention must be easy to apply and readily washed off with water. Barrier creams are advocated as a practical means to prevent the occurrence of irritant as well as allergic dermatitis. The invisible protective effect produced when the protective composition is applied prevents access to the skin and penetration of the skin of noxious substances. The compositions of the invention other than for the express addition of a surfactant as taught herein are generally known and their formulation will be understood by those skilled in the art for such vehicles in formulating topical compositions. Still further examples of such known vehicles include the following:

### Lotion

Water

Sodium lactate

Glycerin

Urea

Glyceryl stearate

C12-15 alkyl benzoate

Octyl dodecanol

Cyclomethicone

Cetyl alcohol

Tocopheryl acetate

Xanthum gum

### Ointment

White petrolatum

Octyl dodecanol

Glycerol mono-di-& trioleate

Glycerin

Oleic acid

Propylene glycol

Water

### Gel

(a) Water

Carbomor 934P

Glycerin

Disodium EDTA

Propylene glycol

Sorbic acid

PPG—20 methyl glucose ether di stearate

Cyclome thicone and dimethicone copolyol

Benzyl alcohol

Butylated hydroxy toluene

b) butylated hydroxy toluene

Hydroxy propyl cellulose

Alcohol (denatured with tert. butyl alcohol

Brucine sulfate

Water

### Cream

a) Stearic acid

Isopropyl myristate

Polyoxyl 40 stearate

Xanthum gum

Ascorbic acid

Butzlated hydroxy toluene

Water

b) White petrolatum

Isopropyl myristate

Lanolin alcohols

Mineral oil

Ceto stearyl alcohol

Aluminum stearate

Edetate disodium

Lactic acid

Magnesium stearate

Water

It was not known heretofore to combine the barrier protectant composition with a surfactant and preferably a lathering surfactant to facilitate removal of the barrier composition. The surfactant in addition to its usual properties as a surface active agent, i.e. lowering the surface tension of liquids, will add an exfoliating and cleansing action to the preparation and will optimize the consistent deposition of the bioactive agents and the conditioning agents if the latter are present. If a lathering surfactant is used, it is a surfactant which when combined with water and then agitated, generates a foam or lather. The surfactant facilitates the removal of the cream, gel, or film only, water being required. The conditioning component is useful for providing a conditioning benefit to the skin during the use of a hair dye, hair relaxant, hair waving composition and the like. Conditioners are largely positively charged (cationic) molecules that neutralize negatively charged sites on the skin surface caused by previous damage due to drying and chemicals used on the hair during the beautifying processes. The conditioning component of these protective compositions comprises about 1% to about 25% of the protective composition. Nonlimiting examples of conditioning agents useful as oil soluble conditioning agents include those selected from the group consisting of mineral oil, petrolatum ethylene glycol, propylene glycol, cyclomethicones having from 3 to 9 silicon atoms, vegetable oils, polypropyleneglycol e.t.c. and mixtures of these conditioning agents. Nonlimiting examples of conditioning agents useful as water soluble conditioning agents include those selected from the group consisting of polyhydric alcohols, polypropylene glycols, polyethylene glycols, urea, pyrolidone carboxylic acid e.t.c. Specific examples of useful water soluble conditioning agents include material such as urea, guanidine, glycolic acid and glycolate salts (e.g. ammonium and quaternary alkyl ammonium); lactate acid and lactate salts, sucrose, fructose sorbitol, mannitol glycerol, polyethelene glycols, polypropylene glycols hyaluronic acid and mixtures there of. Lathering surfactants as used in these compositions are those which when combined with water and agitated generate a foam sufficient to lather. For use in these compositions the selected surfactant should be mild. A wide variety of lathering surfactants are useful herein and include those selected from the group consisting of anionic lathering surfactants, nonionic lathering surfactants and amphotheric lathering surfactants, and mixtures thereof. These surfactants can be used in amounts ranging from 1% to about 10%. A wide variety of anionic lathering surfactants are useful; examples include those selected from the group consisting of sarcosinates, sulfates, isethionates, taurates, phosphates, lactylates, glutamates Amongst the isethionates. The alkoyl isethionates are preferred, and amongst the sulfates, the alkyl and alkyl ether sulfates are preferred. Other anionic materials include alkanoyl sarosinates such as ammonium, sodium, potassium and alkanolamine (e.g., triethanolamine), a preferred examples of which are sodium lauroylsarcosinate, sodium cocoyl sarcosinate, ammonium lauroyl sarcosinate etc. TEA salts of sarcosinates are also useful. Nonlimiting examples, of preferred anionic lathering surfactants useful herein include those selected from the group consisting of sodium lauryl sulfate, ammonium laureth sulfate, sodium laureth sulfate, ammonium cetyl sulfate, ammonium cocoyl isothiocynate, sodium lauroyl isethionate, sodium myristoyl sarcosinate, sodium lauroyl lactylate etc. Especially preferred for use herein is ammonium lauryl sulfate, ammonium laureth sulfate, sodium lauryl sarcosinate, sodium cocoyl sarcosinate, sodium myristoyl sarcosinate, sodium lauroyl lactylate, and triethanolamine lauroyl lactylate. Nonionic lathering surfactants useful herein include those selected from the group consisting of alkyl glucosides, alkyl polyglucoside polyhydroxy fatty acid amides, alkoxylated fatty acid esters, lathering sucrose esters, amine oxides and mixtures thereof, Such as alkyl glucosides and alkyl polyglucosides. A wide variety of amphoteric lathering surfactants, can be used in the compositions. Particularly useful are those which are broadly described as derivatives of aliphatic secondary and tertiary amines, preferably wherein the nitrogen is in a cationic state, in which the aliphatic radicals can be straight or branched chains or branched chain and wherein one of the radicals contains an ionizable group, e.g., carboxy, sulfonate, sulfate, phosphate, or phosphate. Nonlimiting examples of amphoteric or zwitterionic surfactants are those selected from the group consisting of betaines, sultaines, hydroxysultaines, alkyliminoacetates, iminodialkanoates, aminoalkanoates, and mixtures thereof. Examples of betaines include the higher alkyl betaines, such as coco dimethyl carboxymethyl betaine, lauryl dimethyl alphacarboxyethyl betaine, cetyl dimethyl carboxymethyl betaine, cetyl dimethyl betaine etc. Examples of sultaines and hydroxysultaines include materials such as ococamidopropyl hydroxysultane The composition of the present invention can also comprise one or more cationic surfactants, provided these materials do no interfere with the overall lathering characteristics of the lathering surfactants. Cationic surfactants are useful as emulsifiers. Nonlimiting examples of cationic surfactants useful herein include cationic alkyl ammonium These cationic emulsifiers include stearamidopropyl PG-dimonium chloride phosphate, stearamidopropyl etyldimonium ethosulfate, etc, Examples of quaternary ammonium salt cationic surfactants include those selected from the group consisting of cetyl ammonium chloride, cetyl ammonium bromide, lauryl ammonium chloride, lauryl ammonium bromide, stearyl ammonium chloride, stearyl ammonium bromide etc. Preferred cationic surfactants useful herein include those selected from the group consisting of dilauryl dimethyl ammonium chloride, distearyl dimethyl ammonium chloride, dimyristyl dimethyl ammonium chloride, dipalmityl dimethyl ammonium chloride, distearyl dimethyl ammoniujm chloride and mixtures thereof. Further and more complete listings of surfactants useful in the protective composition of this invention are disclosed in McCutcheon's , Detergents and Emulsifiers, North American edition (1986) published by Allured Publishing Corporation and McCutheion's Functional Materials (1992). The weight ratio of the surfactant to the conditioning component when present is preferablyl 1:1. The composition may contain one or more polymers, preferably cationic polymers, nonionic polymers and mixtures thereof. Examples of such polymers useful herein include, but are not limited to gums, hydrophilic colloids, biological polymers and proteins and mixtures there of. Examples of these materials include carboxymethyl hydroxy carboxymethylcellulose, carboxymethylhydroxypropyl guar, carrageenan, cellulose gum, gelatin, guar gum, hydroxyethylcellulose, hydroxypropyl cellulose etc.

To any of the protective compositions, one or combinations of several biologic agents may be incorporated. These are both to protect as well to enhance the physiologic condition of the skin to which the composition is being applied. Such agents could include vitamins, proteins (hydrolyzed), complex carbohydrates such as hyaluronic acid, antioxidants and plant products. Examples of such biologically active agents include: Vitamins A, B6, C, D2 and D3, E, panthenol, aloe vera, grape seed extract, hydrolyzed casein, hydrolyzed collagen, and a wide variety of other hydrolyzed animal and vegetable proteins. The hydrolyzed proteins should be in segments having molecular weights from 1000 to 5000. Complex mucopolysaccharides and some of the products produced by hydrolysis of the mucopolysaccharides e.g. hyaluronic acid, chondroitin sulfuric acid and their constituent amino sugars (glucosamine and galactosamine) All of the above agents have a wide variety of pharmacologic actions. These include antioxidant activity, reduction of inflammation, and improvement and extension of cellular viability.

I claim

1. A skin protective composition for topical use that can prevent contact and passage to the skin of toxic chemicals and irritants found in the cosmetic industry comprising a cosmetic vehicle or base and about 0.5 to about 25% by weight of a surfactant, said composition being readily removable with water.

2. A skin protective composition according to claim 1 wherein said cosmetic vehicle or base is a member selected from the group consisting of gels, creams, ointments, aerosol sprays and lotions.

3. A skin protective composition according to claim 1 wherein said vehicle or base contains at least one lipid substantially insoluble in water.

4. A skin protective composition according to claim 1 wherein said surfactant is a member selected from the group consisting of anionic, nonionic and amphoteric surfactants.

5. A skin protective composition according to claim 1 wherein said surfactant is a lathering surfactant.

6. A skin protective composition according to claim 1 containing at least one member selected from the group consisting of conditioners, emollients, lubricants, protectants, antioxidants, vitamins, proteins, complex carbohydrates and plant derived products.

7. A skin protective composition according to claim 1 in the form of a cream.

8. A skin protective composition according to claim 1 in the form of a gel.

9. A skin protective composition according to claim 1 in the of a lotion.

10. A skin protective composition according to claim 1 in the form of an aerosol spray.

11. A skin protective composition according to claim 1 in the form of an ointment.

12. A skin protective composition according to claim 1 in the form of a liquid which dries to form a film on the skin.

13. A skin protective composition according to claim 3 wherein said lipid is a member selected from the group consisting of petrolatum, C 10-30 cholestrol/lanosterol esters, beeswax, polyethylene wax, paraffin, triglyceryl mono stearate, decaglyceryl stearate and polyglyceryl stearate.

14. Method of manufacturing the composition according to claim 1 comprising the step of adding at least one surfactant into the cosmetic base or vehicle.

15. Method according to claim 14 wherein said surfactant is added in an amount of about 0.5 to about 25% by weight.

16. Method according to claim 15 wherein said surfactant is a foaming surfactant.

17. A method for eliminating or preventing contact and passage to the skin of toxic chemicals and irritants found in the cosmetic industry, comprising the steps of applying to the skin in the area where contact of the toxic chemicals and irritants is to be avoided an effective amount of the composition of claim 1.

18. A method according to claim 17 comprising the further step of wetting with water said applied composition for removing said composition.
